(19) United States
(12) Patent Application Publication
(10) Pub. No.: US 2021/0052765 A1
(43) Pub. Date: Feb. 25, 2021

(54) AIR CLEANER SYSTEM

(71) Applicant: ECLAIR, LTD., Tokyo (JP)

(72) Inventor: Masakazu GOTO, Tokyo (JP)

(21) Appl. No.: 16/641,427

(22) PCT Filed: Jul. 24, 2018
(86) PCT No.: PCT/JP2018/027732

§ 371 Date: Feb. 24, 2020

(30) Foreign Application Priority Data
Sep. 19, 2017 (JP) 2017-179018

(51) Int. Cl. A61L 9/22; A61L 9/015; H01T 23/00; H01T 19/02; F24F 3/16

(52) U.S. Cl. CPC A61L 9/22; A61L 9/015; A61L 2209/111; H01T 19/02; F24F 3/16; H01T 23/00

(57) ABSTRACT

An air-purifier system is provided that generates an ionic wind that is comfortable for the persons in a space to obtain a comfortable environment. The air-purifier system includes an air purifier having a first electrode that has first electrode-structures where each has a first conductive area that is substantially shaped as a circular ring between a first hollow and second hollow, a second electrode that has second electrode-structures where each has a third hollow that is larger in diameter than the first hollow and is substantially shaped as a circle between a third hollow and a fourth hollow a holder to fix the electrodes with a gap therebetween, and a third electrode that has third electrode-structures where each extends along the central axis of the first and the second electrode-structure; a sensor that measures environmental data in a space where the air purifier is provided; and a controller that controls the operations of the air purifier based on the environmental data that are measured by the sensor.

### TECHNICAL FIELD

The present invention relates to an air-purifier system that generates an ionic wind that is comfortable for persons in the space where the ionic wind is generated.

### BACKGROUND ART

Conventionally, a device has been known that generates, by a corona discharge, an ionic wind that includes ozone in a low concentration. By a conventional device a corona discharge is generated between a needle-like electrode and a main circular electrode formed in a plane and a subsidiary circular electrode that surrounds the main circular electrode, to generate an ionic wind. By such a device, an ionic wind is generated by the corona discharge, but increasing the volume of the ionic wind has been required. Thus, an air purifier was proposed that has a plurality of electrodes that generates a large volume of the ionic wind, that can be easily handled, and that can be easily maintained (see Patent Literature 1).

Though the volume of the ionic wind is increased by the invention of Patent Literature 1, there is a possibility that an excessive volume of the ionic wind may cause the smell of ozone to be generated so that persons near it would be unpleasantly affected by that smell.

Thus, the present invention aims to provide an air-purifier system that utilizes an air purifier that generates a large volume of the ionic wind that is comfortable to persons in such a space, i.e., making the environment comfortable.

### PRIOR-ART PUBLICATION

### Patent Literature

### [Patent Literature 1]

Japanese Registered Utility Model No. 3210591

### SUMMARY OF INVENTION

To solve the above-mentioned problem, an air-purifier system of a first aspect of the present invention comprises, for example, as in FIGS. 1, 2, 3, and 8, an air purifier 1, a sensor 110 that measures environmental data obtained in a space 100 where the air purifier 1 is provided, and a controller 120 that controls operations of the air purifier 1 based on the environmental data that are measured by means of the sensor 110. The air purifier 1 has a first metal layer 10 that is a first metal body that is plate-like, that has a first pattern of hollows, and to which a first voltage is applied. It also has a second metal layer 20 that is a second metal body that is plate-like, that has a second pattern of hollows that differs from the first pattern of hollows, to which a second voltage is applied, and that is placed vertically above the first metal layer 10. It also has an electrode 38 to which a third voltage that differs from the first and second voltage is applied and that is located at a central axis of the hollows of the first and second patterns. The first pattern of hollows includes first and second hollow areas 12, 14 around the central axis. The first metal layer 10 has a first metal area 16 between the first and second hollow areas 12, 14. The second pattern of hollows includes third and fourth hollow areas 22, 24 around the central axis. The second metal layer 20 includes a second metal area 26 between the third and fourth hollow areas 22, 24. Corona discharges are generated between the first metal layer 10 and the electrode 38 and between the second metal layer 20 and the electrode 38, to generate a wind with ions and ozone.

By this configuration, since the air purifier is utilized that generates a large volume of the ionic wind by generating the corona discharges between the first metal layer and the electrode and between the second metal layer and the electrode, and since the controller controls the operations of the air purifier based on the environmental data obtained in a space where the air purifier is provided, the air-purifier system can generate an ionic wind that is comfortable for the persons in the space and can provide a comfortable environment. Here, the environmental data obtained in a space denotes data that relate to the environment in the space, such as an odor, a number of various germs, a density of ozone, PM2.5 (particulate matter less than 2.5 microns in diameter), a density of particles that include an amount of floating pollen, etc. The operations of the air purifier denote the activation and deactivation of the air purifier, the output of it, i.e., the volume of the ionic wind that includes ozone generated by it, etc.

To solve the above-mentioned problem, an air-purifier system of a second aspect of the present invention comprises, for example, as in FIGS. 1, 2, 3, and 8, an air purifier 1, a sensor 110 that measures environmental data obtained in a space 100 where the air purifier 1 is provided, and a controller 120 that controls operations of the air purifier 1 based on the environmental data that are measured by means of the sensor 110. The air purifier 1 has a first electrode 10 that is a conductive plate and a plurality of first electrode-structures 18, each of which has a first hollow 12 that is substantially shaped as a circle and second hollows 14 that are substantially shaped as a circular ring that have a common central axis with the first hollow and has a first conductive area 16 that is substantially shaped as a circular ring between the first hollow 12 and the second hollows 14. The air purifier 1 also has a second electrode 20 that is a conductive plate and a plurality of second electrode-structures 28, each of which has a third hollow 22 that is substantially shaped as a circle that is larger in diameter than the first hollow 12 and fourth hollows 24 that are substantially shaped as a circular ring that has a common central axis with the third hollow 22 and has a second conductive area 26 that is substantially shaped as a circular ring between the third hollow 22 and the fourth hollows 24. The second electrode 20 is placed vertically above the first electrode 10 so that the second electrode-structures 28 has a common central axis with the first electrode-structures 18. The air purifier 1 also has a means 60 for fixing the electrodes that fixes the first electrode 10 and the second electrode 20 so that they have a gap between them. The air purifier 1 also has a third electrode 30 that has a plurality of third electrode-structures 38 that are elongated on the respective central axes of the first electrode-structures 18 and the second electrode-structures 28 and are separate from the first electrode-structures 18 and the second electrode-structures 28. The third electrode 30 is located so that the first electrode-structures 18, the second electrode-structures 28, and the third electrode-structures 38, are arranged in this order. The third electrode 30 also has a third electrode-plate 32 that secures distal ends 37 of the third electrode-structures 38 that are separate from the second electrode-structures 28. Corona discharges are generated between the first electrode-structures 18 and the third electrode-structures 38 and between the second electrode-structures 28 and the third electrode-structures 38 by applying a negative voltage to the first electrode 10 and the second electrode 20 and a positive voltage to the third electrode 30, to generate an ionic wind that flows from the first electrode-structures 18 and the second electrode-structures 28 in the direction away from the third electrode-structures 38.

By this configuration, since an air purifier is utilized that generates a large volume of the ionic wind from the first electrode-structures and from the second electrode-structures in the direction away from the third electrode-structures by generating the corona discharges between the first electrode-structures and the third electrode-structures and between the second electrode-structures and the third electrode-structures, and since the controller controls the operations of the air purifier based on the environmental data obtained in the space where the air purifier is provided, the air-purifier system can generate the ionic wind that is comfortable for the persons in the space and can provide a comfortable environment.

By the air-purifier system of a third aspect of the present invention, for example, as in FIG. 8, in the air-purifier system 90 of the first or second aspect the environmental data obtained in a space are at least one of an odor, a number of various germs, and a density of ozone. By this configuration, since the operations of the air purifier are controlled based on an odor, a number of various germs, or a density of ozone in a space, the air-purifier system provides an environment that is comfortable for the persons in the space.

By the air-purifier system of a fourth aspect of the present invention, for example, as in FIG. 8, in the air-purifier system 90 of any of the first, second and third aspects the controller 120 receives data on an environment through a public line 202 and controls the operations of the air purifier 1 based on the data on the environment. By this configuration, since the controller receives the data on the environment through a public line and controls the operations of the air purifier based on the data on the environment, the operations of the air purifier can be controlled based on information on the environment outside of the space where the air purifier is provided, such as a forecast on floating PM2.5 or pollen. Thus, the air-purifier system provides an environment that is comfortable for the persons in the space even if the environment outside the space changes.

By the air-purifier system of a fifth aspect of the present invention, for example, as in FIG. 8, in the air-purifier system 90 of any of the first to fourth aspects the environmental data obtained in a space that have been measured by means of a sensor 110 are transmitted to the controller 120 through a radio communication. By this configuration, since the sensor is connected to the controller by means of the radio communication, the location of the sensor is flexible, to be easily placed at a proper position.

By the air-purifier system of a sixth aspect of the present invention, in the air-purifier system 90 of any of the first to fifth aspects the controller 120 has a timer function for the air purifier 1. By this configuration, since the controller has the timer function for the air purifier, the air purifier can be operated when nobody is in the space or when the environment in the space deteriorates, e.g., when an odor is generated. Thus, the air-purifier system provides an environment that is comfortable for the persons in the space.

By the air-purifier system of a seventh aspect of the present invention, in the air-purifier system 90 of any of the first to sixth aspects the controller 120 controls the operations of the air purifier 1 by adjusting a voltage to the air purifier 1. By this configuration, since the operations of the air purifier are controlled by adjusting the voltage to the air purifier, the air-purifier system provides an environment that is comfortable for the persons in the space by adjusting the operations of the air purifier.

By the air-purifier system of an eighth aspect of the present invention, for example, as in FIG. 7, in the air-purifier system 90 of any of the first to seventh aspects the air purifier 1 moves at least one of the first metal layer or the first electrode 10, the second metal layer or the second electrode 20, and the electrode that is located at the central axis of the hollows of the first and the second patterns or the third electrode 30, to change a distance between the first electrode 10 and the third electrode 30 or between the second electrode 20 and the third electrode 30 or a distance between the first metal layer and the electrode that is located at the central axis of the hollows of the first and second patterns or between the second metal layer and the electrode that is located at the central axis of the hollows of the first and second patterns, to control the operations of the air purifier 1. By this configuration, since the operations of the air purifier, namely, the volume of the ionic wind that is generated, can be controlled by changing a distance between the electrodes, the air-purifier system provides an environment that is comfortable for the persons in the space by adjusting the operations of the air purifier. Especially, since the distance between the electrodes is changed, the operations can be more easily controlled compared to adjusting the voltage.

By the air-purifier system of a ninth aspect of the present invention, for example, as in FIGS. 1 and 4, in the air-purifier system 90 of any of the second aspect, and the third to eighth aspects that refer to the second aspect, the air purifier 1 further has a fourth electrode 40 that is a conductive plate and that has a plurality of fourth electrode-structures 48, each of which has a fifth hollow 42 that is substantially shaped as a circle and that is larger in diameter than the third hollow 22 and a conductive area at a rim of the fifth hollow 42. The fourth electrode 40 is placed vertically above the first electrode 10 and above the second electrode 20 and between the second electrode 20 and the third electrode 30 so that the fourth electrode-structures 48 have common central axes with the first electrode-structures 18 and the second electrode-structures 28. The fourth electrode 40 is fixed by the means 60 for fixing the electrodes. A negative voltage is applied to the fourth electrode 40. By this configuration, since a corona discharge is generated between the fourth electrode-structure and the third electrode-structure to generate an ionic wind from the fourth electrode-structure in the direction away from the third electrode-structure, the air purifier generates a large volume of the ionic wind.

By the air-purifier system of a tenth aspect of the present invention, for example, as in FIG. 7, in the air-purifier system 90 of the ninth aspect the air purifier 1 moves the fourth electrode 40 to control the operations of the air purifier 1 by changing a distance between the fourth electrode 40 and the third electrode 30. By this configuration, since the volume of the ionic wind that is generated in the air purifier is controlled by changing the distance between the fourth electrode and the third electrode, the air-purifier system provides an environment that is comfortable for the persons in the space.

By the air-purifier system of an eleventh aspect of the present invention, for example, in the air-purifier system 90 of any of the first to tenth aspects, the controller 120 generates an alarm when the environmental data obtained in the space are outside a scope of a predetermined range. By this configuration, since the controller generates an alarm when the environmental data obtained in the space are outside the scope of the predetermined range, attention is called to an abnormal environment, to keep persons safe. Herein, the words “generates an alarm” denote that the controller displays an alarm, that the controller sounds an alarm, or that the controller sends an alarm signal to some other device.

By the air-purifier system of a twelfth aspect of the present invention, for example, as in FIG. 7, in the air-purifier system 90 of any of the first to eleventh aspects the air purifier 1 further comprises an aromatic substance 80 that emits an aromatic odor when the aromatic substance is warmed. By this configuration, since it smells good and thus the smell of ozone is suppressed during the operation of the air purifier, the air-purifier system provides an environment that is comfortable for the persons in the space.

The air-purifier system of a thirteenth aspect of the present invention, for example, as in FIG. 8, in the air-purifier system 90 of any of the first to twelfth aspects, comprises a plurality of the air purifiers 1. By this configuration, since the air-purifier system has a plurality of the air purifiers, the air-purifier system provides an environment that is comfortable for the persons in the space by generating a large volume of the ionic wind by means of the air purifiers. Especially, since the ionic winds that correspond to the environmental data that differ for each place in the space can be generated by means of the air purifiers, the air-purifier system provides a uniform environment that is comfortable for the persons even in a large space.

The present invention can provide an air-purifier system that utilizes an air purifier that generates a large volume of the ionic wind and that generates a volume of the ionic wind that is comfortable to persons in such a space, i.e., making the environment comfortable.

The basic Japanese patent application, No. 2017-179018, filed Sep. 19, 2017, is hereby incorporated by reference in its entirety in the present application.

The present invention will become more fully understood from the detailed description given below. However, the detailed description and the specific embodiments are only illustrations of the desired embodiments of the present invention, and so are given only for an explanation. Various possible changes and modifications will be apparent to those of ordinary skill in the art on the basis of the detailed description.

The applicant has no intention to dedicate to the public any disclosed embodiment. Among the disclosed changes and modifications, those which may not literally fall within the scope of the present claims constitute, therefore, a part of the present invention in the sense of the doctrine of equivalents.

The use of the articles “a,” “an,” and “the” and similar referents in the specification and claims are to be construed to cover both the singular and the plural form of a noun, unless otherwise indicated herein or clearly contradicted by the context. The use of any and all examples, or exemplary language (e.g., “such as”) provided herein is intended merely to better illuminate the invention, and so does not limit the scope of the invention, unless otherwise stated.

### BRIEF DESCRIPTION OF DRAWINGS

FIG. 1 is a sectional view of the electrodes of the air purifier.

FIG. 2 is a plan view of the first electrode.

FIG. 3 is a plan view of the second electrode.

FIG. 4 is a plan view of the fourth electrode.

FIG. 5 is a plan view of the first, second, and fourth electrodes, which are vertically placed one above the other and fixed by the means for fixing the electrodes.

FIG. 6 is a perspective view of the third electrode.

FIG. 7 is a sectional view of a major part of the air purifier.

FIG. 8 is a block diagram of the air-purifier system, which is an embodiment of the present invention.

### DESCRIPTION OF EMBODIMENTS

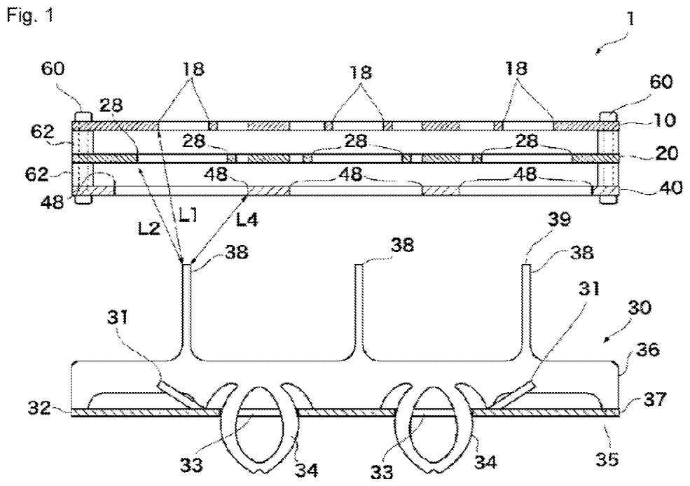

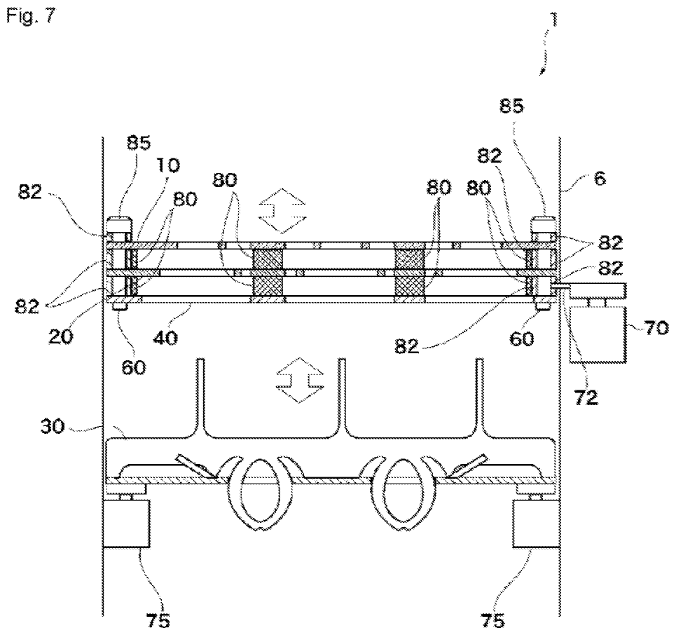

Below, embodiments of the present invention are discussed with reference to the drawings. The members that are identical or that correspond to each other are denoted by the same reference number. So a duplicate explanation is omitted. FIG. 1 illustrates a sectional view of a first electrode 10, a second electrode 20, a fourth electrode 40, and a third electrode 30, of an air purifier 1. The air purifier 1 has the first electrode 10, the second electrode 20, and the fourth electrode 40, which are placed vertically one above the other and are fixed by a means 60 for fixing the electrodes. It also has the third electrode 30, which faces these electrodes. It also has a power source (not shown) for applying a negative voltage to the first electrode 10, the second electrode 20, and the fourth electrode 40 and a positive voltage to the third electrode 30. It also has a casing 6 (see FIG. 7) that houses and secures these members and that takes in the outside air and discharges an ionic wind. It also has a switch (not shown) to activate and deactivate the air purifier 1. Incidentally, the air purifier 1 may be activated and deactivated by means of a controller 120, which is discussed below and may have no switch. In the air purifier 1 the ionic wind that has been generated by the electrodes is discharged through a nozzle that is formed in the casing 6.

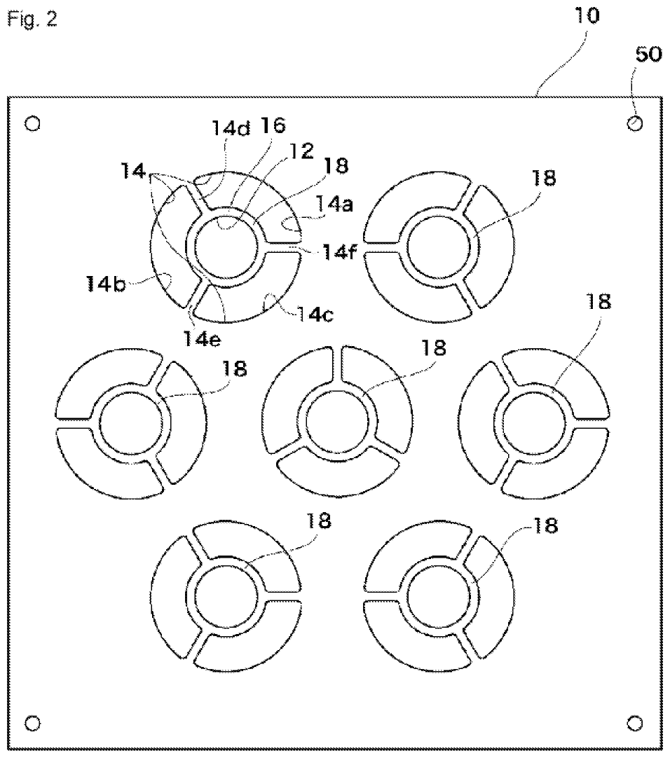

FIG. 2 illustrates a plan view of the first electrode 10. The first electrode 10 is formed by a plate of a conductive material. The plate of a conductive material is typically a metal plate of iron, copper, aluminum, etc., but may be formed by a conductive ceramic, etc. In the first electrode 10 a plurality of first electrode-structures 18 are formed. Specifically, the first electrode-structures 18 are formed as follows. A first hollow 12 that is substantially shaped as a circle and the second hollows 14 that are substantially shaped as a circular ring are concentrically formed. If the first hollow 12 is shaped as a circle, the distances from a third electrode-structure 38 become uniform so that a uniform corona discharge can be obtained, and so the ionic wind becomes uniform. However, in so far as a corona discharge is obtained, the first hollow 12 may be shaped as a polygon, an ellipse, the shape of an onigiri, etc. The second hollows 14 are formed to be concentric with the first hollow 12 so that a first conductive area 16 is formed around the first hollow 12. Here, what is termed “concentric” need not be strictly concentric, but may be misaligned in so far as the first conductive area 16 is formed. The second hollows 14 may be formed by, for example, three segment-shaped hollows 14a, 14b, 14c, and connecting parts 14d, 14e, 14f, that are parts between the segment-shaped hollows 14a, 14b, 14c, and which parts support the first conductive area 16. The number of segment-shaped hollows and connecting parts is not limited to three, but may be two or four or more. Since the first conductive area 16 is formed in this way, it functions, especially its inner edge, as the first electrode-structure 18 that generates the corona discharge with the third electrode-structure 38.

The first electrode 10 has seven first electrode-structures 18. Since a plurality of first electrode-structures 18 are formed, a large volume of the ionic wind can be generated by many corona discharges. The seven first electrode-structures 18 are located at equal distances from each other. By locating them in this way a uniform ionic wind can be obtained. The number of first electrode-structures 18 is not limited to seven, but may be arbitrarily changed based on a required volume of the ionic wind. In the first electrode 10 through-holes 50 to be used for the means for fixing the electrodes are formed at four corners.

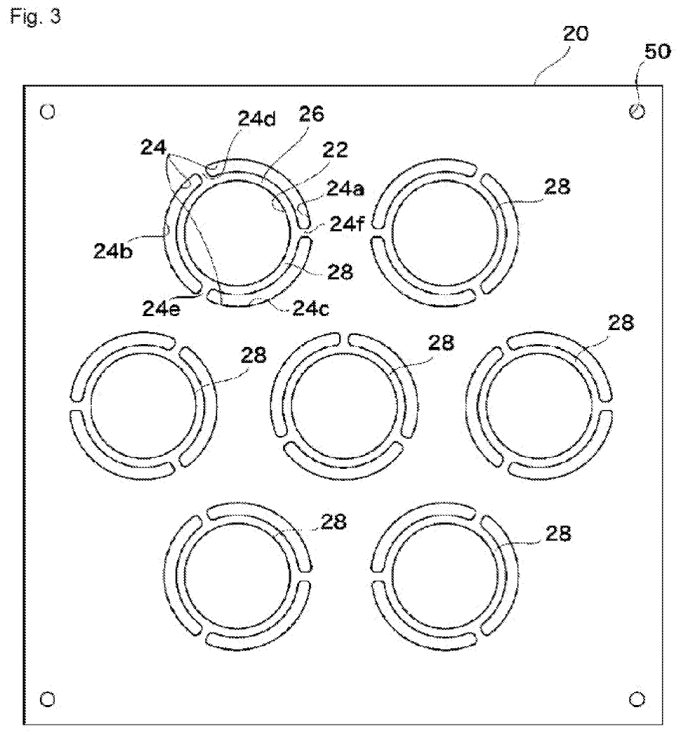

FIG. 3 illustrates a plan view of the second electrode 20. Since the second electrode 20 is similar to the first electrode 10, only the different points are discussed, and so a duplicate explanation is omitted. In the second electrode 20 a plurality of second electrode-structures 28 are formed. Specifically, the second electrode-structures 28 are formed as follows. A third hollow 22, which is substantially shaped as a circle and corresponds to the first hollow 12, and fourth hollows 24, which are substantially shaped as a circular ring and correspond to the second hollows 14, are concentrically formed. The third hollow 22 is larger in diameter than the first hollow 12. And the second electrode-structures 28 are larger in diameter than the first electrode-structures 18. The fourth hollows 24 may be formed by, for example, three segment-shaped hollows 24a, 24b, 24c, and connecting parts 24d, 24e, 24f, that are parts between the segment-shaped hollows 24a, 24b, 24c, and which parts support a second conductive area 26. The outside diameter of the fourth hollows 24 is the same as that of the second hollows 14, but is not limited to the same. By making the outside diameters the same, the pathway of the ionic wind that is generated by the corona discharge becomes smooth.

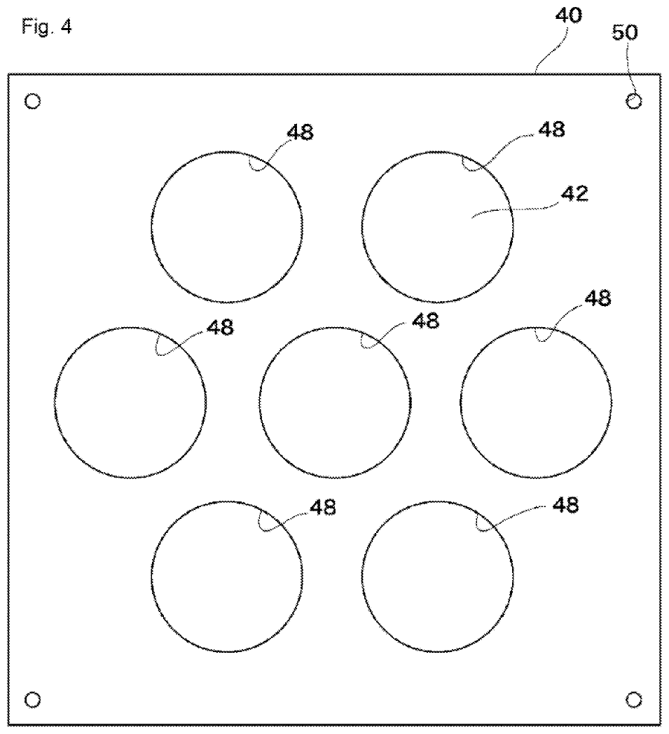

FIG. 4 illustrates a plan view of the fourth electrode 40. Since it is similar to the first electrode 10 and the second electrode 20, only the different points are discussed. So a duplicate explanation is omitted. In the fourth electrode 40 a plurality of fourth electrode-structures 48 are formed. Specifically, the fourth electrode-structures 48 are formed as follows. A fifth hollow 42 that is substantially shaped as a circle is formed. The fifth hollow 42 is the same in diameter as the second hollows 14 and the fourth hollows 24. A conductive area at the rim of the fifth hollow 42 is the fourth electrode-structure 48. Namely, the fourth electrode-structure 48 is larger in diameter than the second electrode-structure 28. The outside diameter of the fifth hollow 42 is the same as that of the second hollows 14 and the fourth hollows 24, but is not limited to that diameter. By making the outside diameters of these hollows the same, the pathway of the ionic wind that is generated by the corona discharge becomes smooth.

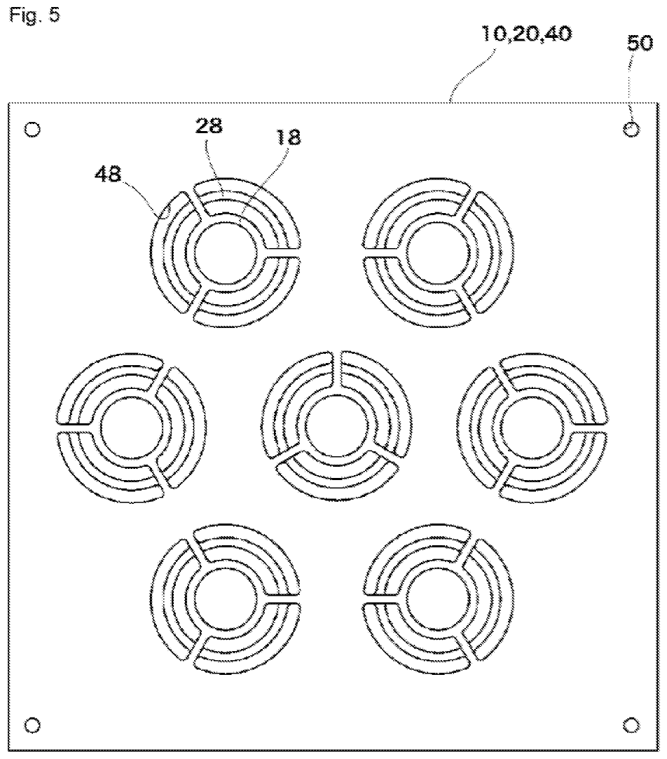

FIG. 5 illustrates a plan view of the first electrode 10, the second electrode 20, and the fourth electrode 40, which are placed vertically one above the other. The view is taken from the first electrode 10 (from the top of FIG. 1). In this embodiment, the first electrode 10, the second electrode 20, and the fourth electrode 40, are placed vertically one above the other so that the seven first electrode-structures 18, the seven second electrode-structures 28, and the seven fourth electrode-structures 48, are concentric. As in FIG. 1, the first electrode 10, the second electrode 20, and the fourth electrode 40, which are placed vertically one above the other, are fixed by the means 60 for fixing the electrodes. In other words, in the conductive plates of the first electrode 10, the second electrode 20, and the fourth electrode 40, the through-holes 50 are formed, through which the means 60 for fixing the electrodes passes. The means 60 for fixing the electrodes is caused to pass through the through-holes 50 in the electrodes 10, 20, 40 and both ends of it are fixed by being widened or screwed, to fix the electrodes. The means 60 for fixing the electrodes may be a publicly-known member, such as a hollow pin, a split pin, an eyelet, or a screw and a nut. In so doing, spacers 62 are inserted between the first electrode 10 and the second electrode 20 and between the second electrode 20 and the fourth electrode 40, so that gaps are formed between them. Since the sizes of the gaps are determined by the spacers 62, they are easily changed based on requirements.

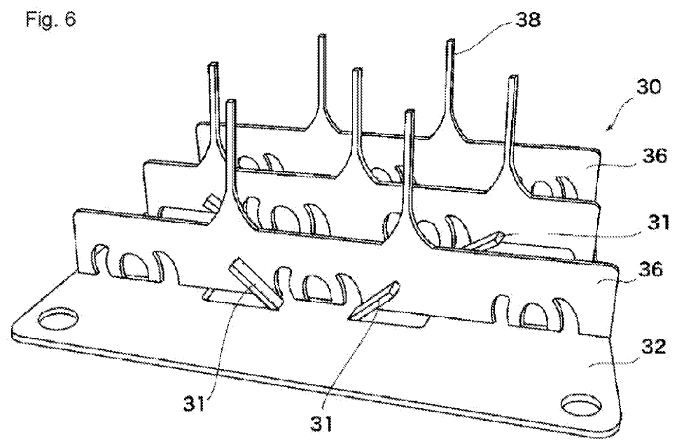

With reference to FIGS. 1 and 6, the configuration of the third electrode 30 is now discussed. The third electrode 30 is located to face the first electrode 10, the second electrode 20, and the fourth electrode 40, to generate the corona discharges between each of them. It has bar-shaped third electrode-structures 38 that are elongated along the central axes of the first electrode-structures 18, the second electrode-structures 28, and the fourth electrode-structures 48. The ends 39 of the third electrode-structures 38, i.e., the proximal ends to the first electrode 10, the second electrode 20, and the fourth electrode 40, are shaped to be flat. Here, what is termed “flat” includes a shape in which the rims of the ends 39 are rounded or beveled or a shape in which the entire ends 39 are shaped as a spheroid (a body that revolves about an axis), which has a smooth surface.

The other ends of the third electrode-structures 38 (the distal ends from the first electrode 10, the second electrode 20, and the fourth electrode 40) are formed by a plate 36. The plate 36 extends in the lateral direction (the left direction and the right direction in FIG. 1) and is integrated with the plate 36 of the neighboring third electrode-structure 38. Namely, in the third electrode 30 as in FIG. 1, three third electrode-structures 38 protrude from one sheet of the plate 36. FIG. 6 also illustrates that two third electrode-structures 38 protrude from one sheet of the plate 36. The number and arrangement of the third electrode-structures 38 can be determined as appropriate based on the numbers and arrangements of the first electrode-structures 18, the second electrode-structures 28, and the fourth electrode-structures 48.

The plate 36 of the third electrode 30 is fixed to the third electrode-plate 32, so that it stands. The third electrode-plate 32 is formed by a conductive plate in the same way as is the first electrode 10, etc. It is placed in parallel to the first electrode 10, etc. The plate 36 has a part 35 that abuts the third electrode-plate 32 at the distal end 37 of the third electrode-structure 38 from the first electrode 10, etc. It also has a part 34 that extends over the distal end 37 to pass through the third electrode-plate 32. The part 34 that passes through the conductive plate is shaped as a C with an open bottom or as a ring, and is thin. That is, the part shaped as a C or as a ring can be elastically and narrowly deformed. Thus, the part shaped as a C or as a ring that has the largest diameter (in the right direction and the left direction in FIG. 1) is in a portion that has passed through the third electrode-plate 32. In the third electrode-plate 32 a through-hole 33 is formed through which the part 34 passes. The diameter (in the right direction and the left direction in FIG. 1) of the through-hole 33 is the same as, or slightly smaller than, the largest diameter of the part 34. Thus, when the part 34 passes through the through-hole 33 it is fixed to it by means of the elasticity of the part 34. And a force is generated in the direction to lift up the third electrode-plate 32. Thus the plate 36 clamps the third electrode-plate 32 between the part 35 and the part 34 to be firmly fixed to the third electrode-plate 32.

As in FIG. 6, the third electrode-plate 32 has folded parts 31 that are folded along the plate 36. Since the folded parts 31 are formed at both sides of the plate 36, the plate 36 is prevented from tilting away from the plane. In this way, the third electrode-structures 38 and the plate 36 are fixed to the third electrode-plate 32 with a simple structure. Incidentally, the configuration of the third electrode 30 is not limited to the above configuration. The plate 36 may be fixed to the third electrode-plate 32 by any publicly-known method.

The first electrode 10, the first hollow 12, the second hollows 14, and the first conductive area 16, may be called the first metal layer, the first hollow area, the second hollow area, and the first metal area, respectively. The second electrode 20, the third hollow 22, the fourth hollows 24, and the second conductive area 26, may be called the second metal layer, the third hollow area, the fourth hollow area, and the second metal area, respectively. The third electrode-structure 38 may be called the electrode that is located at the central axis of the hollows of the first and second patterns, or just an electrode if it is contextually clear. Incidentally, the first electrode 10, the second electrode 20, the third electrode 30, and so on may be just called electrodes.

As in FIG. 7, the air purifier 1 preferably has a means 70 for moving the first, second, and fourth electrodes or a means 75 for moving the third electrode that changes the distances between the first electrode 10, the second electrode 20, and the fourth electrode 40, and the third electrode 30, to be nearer or farther from each other. The means 70 for moving the first, second, and fourth electrodes has a shaft that is extended or shortened by means of a solenoid. A beam 72 that is fixed to the means 60 for fixing the electrodes is fixed to the tip of the shaft of the means 70 for moving the first, second, and fourth electrodes, to vertically move the means 60 for fixing the electrodes through the beam 72 by vertical movement of the shaft. The means 70 is fixedly supported by the outside of the case 6 of the air purifier 1. The beam 72 passes through the case 6. By changing the distances between the first electrode 10, the second electrode 20, and the fourth electrode 40, and the third electrode 30, to be nearer or farther from each other, the volume of the ionic wind that is generated by the air purifier 1 can be adjusted. Further, by changing the voltage that is applied to the first electrode 10, the second electrode 20, and the fourth electrode 40, and the third electrode 30, that volume can be adjusted. When the voltage is changed it must be within the range to generate a corona discharge. Thus, there is a limit set by the range for changing the voltage. Further, the cost of a transformer increases. Controlling the transformer becomes difficult. It is preferable to change the distances between the electrodes to adjust the volume of the ionic wind, because the distances can be precisely adjusted. Further, by a combination of changing the voltage and changing the distances between the electrodes, the volume of the ionic wind can be widely adjusted by changing the voltage and can be precisely adjusted by changing the distances. Thus, that volume can be widely and accurately adjusted.

The means 70 for moving the first, second, and fourth electrodes is not limited to one in which the shaft is extended or shortened by an electromagnetic force of a solenoid. It may be one in which the shaft is extended or shortened by a publicly-known configuration, such as a motor and a combination of bevel gears, a configuration in which a screw is threaded and engages with a female screw that rotates. The shaft of the means 70 for moving the first, second, and fourth electrodes may be integrated with the means 60 for fixing the electrodes that is extended and the means 70 may be fixed to the inside of the case. Further, the first electrode 10, the second electrode 20, and the fourth electrode 40 may be moved without the means 60 for fixing the electrodes moving. For example, an elastic body such as a spring and an electromagnet that applies an electromagnetic force to the conductive plates of the first electrode 10, the second electrode 20, and the fourth electrode 40, are placed between the conductive plates to move the first electrode 10, the second electrode 20, and the fourth electrode 40. Or, another publicly-known configuration may be used. Further, the first electrode 10, the second electrode 20, and the fourth electrode 40 may each be separately moved by means of a publicly-known configuration. Incidentally, when any of the electrodes is separately moved, no spacer 62 is required. The means 70 for moving the first, second, and fourth electrodes may be supported by the case 6 of the air purifier 1 directly or through another member.

Since the means 75 for moving the third electrode may be a publicly-known configuration as is the means 70 for moving the first, second, and fourth electrodes, a duplicate explanation is omitted. As in FIG. 7, the means 75 for moving the third electrode is located inside the case 6. It may be located outside the case 6, as is the means 70 for moving the first, second, and fourth electrodes. Since the third electrode 30 is not fixed by the means 60 for fixing the electrodes, the shaft of the means 75 for moving the third electrode is fixed to the third electrode-plate 32. The air purifier 1 may have only the means 70 for moving the first, second, and fourth electrodes, or only the means 75 for moving the third electrode, or both means.

Further, the air purifier 1 may have an aromatic substance 80 that emits an aromatic odor when being warmed. A holder 85 that stores the aromatic substance 80, which is liquid, is provided to the top of the means 60 for fixing the electrodes. A fibrous cylinder 82 is wrapped around the means 60 for fixing the electrodes so that the aromatic substance 80 in the holder 85 is sucked by capillary action. The fibrous cylinder 82 may be provided to be framed by the spacer 62 between the electrodes 10, 20, 40, or may be provided around the spacer 62, or may be provided instead of the spacer 62. A fibrous material is provided to the space between the first electrode 10 and the second electrode 20 and outside of the area of the first and second electrode-structures 18, 28. It is connected to the fibrous cylinder 82 to contain the aromatic substance 80. In the same way, a fibrous material is also provided to the space between the second electrode 20 and the fourth electrode 40. When the air purifier 1 is activated, i.e., a voltage is applied to the electrodes 10, 20, 30, 40, their temperatures increase. The aromatic substance 80 that is contained in the fibrous materials in the air purifier 1 is warmed to emit an aromatic odor. The aromatic substance 80 may be located on the first electrode 10 or another space. The aromatic substance may be solid or natural wood. In this case, it is directly placed on the electrodes 10, 20, 40.

Next, the operations of the air purifier 1 are discussed. By applying a negative voltage to the first electrode 10, to the second electrode 20, and to the fourth electrode 40, and a positive voltage to the third electrode 30, corona discharges are generated between the third electrode-structure 38 and the first electrode-structures 18, between the third electrode-structure 38 and the second electrode-structures 28, and between the third electrode-structure 38 and the fourth electrode-structure 48. The same voltage may be applied to the first electrode 10, ti the second electrode 20, and to the fourth electrode 40, but different voltages may be applied to them. When the same one is applied to them, a single terminal is connected to the first electrode 10, to the second electrode 20, and to the fourth electrode 40, which are placed vertically one above the other, so that the structure is simple.

Since the corona discharges are generated, the ionic wind is generated from the first electrode-structures 18, the second electrode-structures 28, and the fourth electrode-structures 48, in the direction away from the third electrode-structures 38 (upward in FIG. 1). In other words, since air is positively ionized at the first electrode-structures 18, the second electrode-structures 28, and the fourth electrode-structures 48, the ionic wind is generated in the direction away from the third electrode-structure 38, to which a positive voltage is applied. Since the ionic wind is generated at the first electrode-structures 18, the second electrode-structures 28, and the fourth electrode-structures 48, i.e., three positions, a large volume of the ionic wind flows. Incidentally, the ionic wind includes a low concentration of ozone.

The distance L1 between the first electrode-structure 18 and the third electrode-structure 38 is preferably greater than the distance L2 between the second electrode-structure 28 and the third electrode-structure 38. The distance L4 between the fourth electrode-structure 48 and the third electrode-structure 38 is preferably less than the distance L2. At the first electrode-structure 18, which has a short diameter, an ionic wind with a high density is generated. At the second electrode-structure 28, which is larger in diameter, an ionic wind with a lower density is generated. At the fourth electrode-structure 48, which is yet larger in diameter, an ionic wind with a yet lower density is generated. However, the longer the distance to the third electrode-structure 38 is, the weaker the corona discharge is. Thus, the density of the ionic wind becomes lower. By setting the distances L1>L2>L4, an ionic wind with a uniform density in its entirety can be generated. Especially, the first electrode-structures 18, the second electrode-structures 28, and the fourth electrode-structures 48, preferably form a paraboloid centering around the third electrode-structures 38. When they so form a paraboloid, an ionic wind with a uniform density can be generated. Incidentally, the distances L1, L2, L4 are not limited to the above, but may be another relationship, or may be the same.

The air purifier 1 has, for example, seven combinations of the third electrode-structures 38 and the first electrode-structures 18, the second electrode-structures 28, and the fourth electrode-structures 48, to discharge a large volume of the ionic wind. Thus, by using the air purifier 1 a large volume of the ionic wind can be obtained by a simple configuration.

By the above discussion, it is seen that the air purifier 1 has the first electrode 10, the second electrode 20, and the fourth electrode 40. However, it need not have the fourth electrode 40. Accordingly, the volume of the ionic wind will decrease. However, an adequate volume of the ionic wind can be generated depending on applications. Further, the air purifier 1 may have a fifth electrode (not shown), a sixth electrode (not shown), and so on, in addition to the first electrode 10, the second electrode 20, and the fourth electrode 40. The same as with the first electrode 10 and the second electrode 20, the electrodes to be added have a plurality of the electrode-structures that have areas of conductive material that are shaped as a ring. They are placed vertically one over the other with the first electrode 10, etc., so that the electrode-structures are concentric with the first electrode-structures 18, etc.

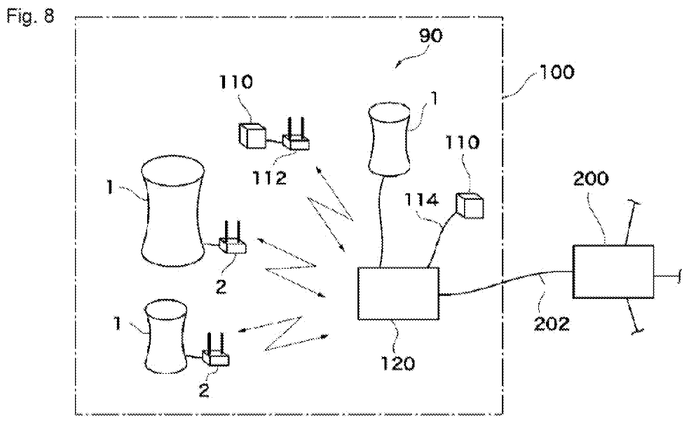

Next, with reference to FIG. 8, the air-purifier system 90 that has the air purifier 1 is discussed. The air-purifier system 90 has the air purifier 1, a sensor 110, and a controller 120. The sensor 110 measures environmental data obtained in the space 100 where the air purifier 1 is located. The controller 120 controls the operations of the air purifier 1 based on the environmental data that are measured by the sensor 110. The space 100 denotes a space where the air purifier 1 is located and where airflow to and from the outside is limited, such as a room, a store, and a warehouse. But, it does not necessarily have a complete wall.

The environmental data include odors, the number of various germs, the density of ozone, and the concentration of particulate matter, such as PM2.5 and pollen. It may include temperature, humidity, the strength and the direction of a wind, and so on. The sensor 110 can be any sensor that measures one of them. In the space 100 a plurality of the sensors 110 may be provided. They may be sensors that measure the same environmental data at different positions or sensors that measure different environmental data.

In so far as the operations of the air purifier 1 can be controlled based on the environmental data that are measured by the sensor 110, the controller 120 may be a controller that controls devices in the space 100 other than the air purifier 1, or a general-purpose PC (personal computer), or another controller.

Signals between the air purifier 1 and the controller 120 and between the sensor 110 and the controller 120 may be transmitted by wire or wireless. The air-purifier system 90 as in FIG. 8 has three air purifiers 1. Two of them transfer signals with the controller 120 wireless through radio communication devices 2 and one of them does so by wire. One of the sensors 110 transfers signals with the controller 120 wireless through a radio communication device 112 and one does so by wire through a cable 114. If the sensor 110 transfers signals with the controller 120 through the radio communication, then positions to place it become flexible to preferably facilitate proper positioning. Incidentally, the sensor 110 may have the radio communication device 112 built-in.

The controller 120 may receive the environmental data from an external system 200 through a public line 202, such as the Internet, to control the operations of the air purifier 1. For example, information on floating PM2.5 or on floating allergic substances in a small amount, such as pollen, may be received from the Japan Meteorological Agency or another agency, to control the operations of the air purifier 1. The controller 120 may have a function to timely activate and deactivate the air purifier 1 as a timer function for the air purifier 1.

Next, the operations of the air-purifier system 90 are discussed. By the air-purifier system 90 the environmental data in the space 100 where the air purifier 1 is located is measured by means of the sensor 110. For example, odors or the number of various germs is measured. When the odors are strong or when the number of various germs is large, the volume of the ionic wind that is generated by the air purifier 1 is preferably increased to enhance the deodorizing power or bactericidal power. Thus, the voltage to be applied to the electrodes 10, 20, 30, 40 of the air purifier 1 is increased or the distance between the first electrode 10, the second electrode 20, or the fourth electrode 40, and the third electrode 30, is narrowed so that the volume of the ionic wind is increased. In contrast, when the odors are weak or when the number of various germs is small, the voltage is decreased or the distance is increased, so that the volume of the ionic wind is decreased. In this way, by controlling the operations of the air purifier 1 based on the environmental data that are measured by the sensor 110, the ionic wind that is comfortable for persons in the space is generated to obtain a comfortable environment without uselessly operating the air purifier 1.

If the environmental data vary in the space, a plurality of the air purifiers 1 and a plurality of the sensors 110 are installed to obtain a more comfortable environment. The plurality of the sensors 110 measure the environmental data at different points and each of the air purifiers 1 operates as appropriate to the position based on the measured data. Thus, when the environmental data differ at points in the space 100, a comfortable environment can be obtained everywhere. For example, such a system is effective in a very large space 100, or in a space 100 in which a factor that changes (deteriorates) the environmental data exists. The air-purifier system 90 may have a single air purifier 1 or a single sensor 110.

Incidentally, the voltages that are applied to the electrodes 10, 20, 30, 40 may be increased or decreased by a transformer (not shown) based on a signal that is transmitted by the controller 120. The air purifier 1 may have the transformer built-in or the controller 120 may function as the transformer.

The sensor 110 may measure the density of ozone. A too high density of ozone may cause the persons in the space 100 to be bothered by the smell of ozone. Thus, if the density of ozone is too high, the volume of the ionic wind that is generated by the air purifier 1 is preferably decreased. To do so, the voltages to be applied to the electrodes 10, 20, 30, 40 are decreased or the distances between the first electrode 10, the second electrode 20, or the fourth electrode 40, and the third electrode 30, is increased, to decrease the volume of the ionic wind. In this way, by controlling the operations of the air purifier 1 based on the environmental data that are measured by the sensor 110, the ionic wind that is comfortable for the persons in the space is generated to obtain a comfortable environment without being bothered by the smell of ozone that is caused by too high a density of ozone.

The controller 120 can control the operations of the air purifier 1 by receiving the environmental data from the external system 200 through the public line 202. For example, if a large amount of floating PM2.5 is expected based on information on floating PM2.5 that is received from the Japan Meteorological Agency, the volume of the ionic wind by the air purifier 1 is preferably increased. This is because PM2.5 will be decomposed by the ionic wind, and will disappear. Thus, the voltages to be applied to the electrodes 10, 20, 30, 40 are increased or the distances between the first electrode 10, the second electrode 20, or the fourth electrode 40, and the third electrode 30, is decreased, to increase the volume of the ionic wind. In this way, by controlling the operations of the air purifier 1 based on the environmental data that are received from the external system 200 through the public line 202, an ionic wind that is comfortable for the persons in the space is generated to obtain a comfortable environment by generating an ionic wind corresponding to the expected change in the environment. In the same way, if a large amount of floating pollen is expected based on information on floating pollen that is received from the Japan Meteorological Agency, the volume of the ionic wind by the air purifier 1 is preferably increased. Fine powder, such as pollen, can be easily oxidized by the ozone so that its mass increases, so as not to float.

Since the controller 120 has the timer function for the air purifier 1, the air purifier 1 is timely activated and deactivated. For example, when the air-purifier system 90 is used in a store, the air purifier 1 is activated after the store is closed and is stopped when it is open, so that the store is deodorized and sterilized, and ozone is prevented from being smelled in the store when customers are in it.

The controller 120 may raise an alarm if the environmental data that are measured by the sensor 110 exceed a predetermined range. Thus, the persons in the space 100 or outside it can be alerted if the environment has deteriorated. For an alarm, for example, the controller 120 may sound an alarm, display an alarm, send a signal to cause an alarm (not shown) inside or outside the space 100 to sound or display an alarm or raise an alarm by another known way.

Since by the air-purifier system 90 the activation and deactivation of and the operations of the air purifier 1, such as the volume of the ionic wind to be generated, are controlled based on the environmental data in the space that are measured by the sensor 110 or on the environmental data that are received from the external system 200 through the public line 202, or by a timer function, persons in the space can obtain an ionic wind that provides a comfortable environment.

For the environmental data other data that affect the environment in the space, such as the temperature, the humidity, the strength and the direction of a wind, may be used. The timer function is not necessarily the activation and deactivation, and may be a function to change the volume of the ionic wind depending on the time of day, or to change the required volume of the ionic wind based on the environmental data depending on the time of day.

Below, the reference signs used in the present specification and the drawings are listed.
1 the air purifier
2 the radio communication device
6 the case
10 the first electrode (the first metal layer)
12 the first hollow (the first hollow area)
14 the second hollows (the second hollow area)
14a, b, c the segment-shaped hollows
14d, e, f the connecting parts
16 the first conductive area (the first metal area)
18 the first electrode-structure
20 the second electrode (the second metal layer)
22 the third hollow (the third hollow area)
24 the fourth hollows (the fourth hollow area)
24a, b, c the segment-shaped hollows
24d, e, f the connecting parts
26 the second conductive area (the second metal area)
28 the second electrode-structure
30 the third electrode
31 the folded part
32 the third electrode-plate
33 the through hole
34 the part that passes through the conductive plate
35 the part that abuts the electrode-plate
36 the plate of the third electrode-structure
37 the distal end of the third electrode-structure
38 the third electrode-structure (the electrode that is located at the central axis of the hollows of the first and second patterns)
39 the tip of the third electrode-structure
40 the fourth electrode
42 the fifth hollow
48 the fourth electrode-structure
50 the through-holes
60 the means for fixing the electrodes
62 the spacer
70 the (first, second, fourth) means for moving the electrode
72 the beam
75 the (third) means for moving the electrode
80 the aromatic substance
82 the fibrous cylinder
85 the holder of the aromatic substance
90 the air-purifier system
100 the space
110 the sensor
112 the radio communication device
114 the cable in the space
120 the controller
200 the external system
202 the public line

1. An air-purifier system comprising:
an air purifier that comprises
a first metal layer that is a first metal body that is plate-like, that has a first pattern of hollows, and to which a first voltage is applied,
a second metal layer that is a second metal body that is plate-like, that has a second pattern of hollows that differs from the first pattern of hollows, to which a second voltage is applied, and that is placed vertically above the first metal layer, and
an electrode to which a third voltage that differs from the first and second voltage is applied and that is located at a central axis of the hollows of the first and second patterns,
wherein the first pattern of hollows includes first and second hollow areas 12, 14 around the central axis,
wherein the first metal layer has a first metal area between the first and second hollow areas,
wherein the second pattern of hollows includes third and fourth hollow areas around the central axis,
wherein the second metal layer includes a second metal area between the third and fourth hollow areas, and
wherein corona discharges are generated between the first metal layer and the electrode and between the second metal layer and the electrode, to generate a wind with ions and ozone;
a sensor that measures environmental data obtained in a space where the air purifier is provided; and
a controller that controls operations of the air purifier based on the environmental data that are measured by means of the sensor.

2. An air-purifier system comprising:
an air purifier 1 that comprises
a first electrode that is a conductive plate and a plurality of first electrode-structures, each of which has a first hollow that is substantially shaped as a circle and second hollows that are substantially shaped as a circular ring that have a common central axis with the first hollow and has a first conductive area that is substantially shaped as a circular ring between the first hollow and the second hollows,
a second electrode that is a conductive plate and a plurality of second electrode-structures, each of which has a third hollow that is substantially shaped as a circle that is larger in diameter than the first hollow and fourth hollows that are substantially shaped as a circular ring that have a common central axis with the third hollow and has a second conductive area that is substantially shaped as a circular ring between the third hollow and the fourth hollows, wherein the second electrode is placed vertically above the first electrode so that the second electrode-structures has a common central axis with the first electrode-structures,
a means for fixing the electrodes that fixes the first electrode and the second electrode so that they have a gap between them,
a third electrode that has a plurality of third electrode-structures that are elongated on the respective central axes of the first electrode-structures and the second electrode-structures and are separate from the first electrode-structures and the second electrode-structures, wherein the third electrode is located so that the first electrode-structures, the second electrode-structures, and the third electrode-structures, are arranged in this order, and wherein the third electrode also has a third electrode-plate that secures distal ends of the third electrode-structures that are separate from the second electrode-structures,
wherein corona discharges are generated between the first electrode-structures and the third electrode-structures and between the second electrode-structures and the third electrode-structures by applying a negative voltage to the first electrode and the second electrode and a positive voltage to the third electrode, to generate an ionic wind that flows from the first electrode-structures and the second electrode-structures in the direction away from the third electrode-structures;
a sensor that measures environmental data obtained in a space where the air purifier is provided; and
a controller that controls operations of the air purifier based on the environmental data that are measured by means of the sensor.

3. The air-purifier system of claim 1, wherein the environmental data obtained in a space are at least one of an odor, a number of various germs, and a density of ozone.

4. The air-purifier system of claim 1, wherein the controller receives data on an environment through a public line and controls the operations of the air purifier based on the data on the environment.

5. The air-purifier system of claim 1, wherein the environmental data obtained in a space that have been measured by means of a sensor are transmitted to the controller through a radio communication.

6. The air-purifier system of claim 1, wherein the controller has a timer function for the air purifier.

7. The air-purifier system of claim 1, wherein the controller controls the operations of the air purifier by adjusting a voltage to the air purifier.

8. The air-purifier system of claim 1, wherein the air purifier moves at least one of the first metal layer or the first electrode, the second metal layer or the second electrode, and the electrode that is located at the central axis of the hollows of the first and the second patterns or the third electrode, to change a distance between the first electrode and the third electrode or between the second electrode and the third electrode or a distance between the first metal layer and the electrode that is located at the central axis of the hollows of the first and second patterns or between the second metal layer and the electrode that is located at the central axis of the hollows of the first and second patterns, to control the operations of the air purifier.

9. The air-purifier system of claim 2, wherein the air purifier further has a fourth electrode that is a conductive plate and that has a plurality of fourth electrode-structures, each of which has a fifth hollow that is substantially shaped as a circle and that is larger in diameter than the third hollow and a conductive area at a rim of the fifth hollow, wherein the fourth electrode is placed vertically above the first electrode and above the second electrode and between the second electrode and the third electrode so that the fourth electrode-structures have common central axes with the first electrode-structures and the second electrode-structures, and wherein the fourth electrode is fixed by the means for fixing the electrodes;
wherein a negative voltage is applied to the fourth electrode.

10. The air-purifier system of claim 9, wherein the air purifier moves the fourth electrode to control the operations of the air purifier by changing a distance between the fourth electrode and the third electrode.

11. The air-purifier system of claim 1, wherein the controller generates an alarm when the environmental data obtained in the space are outside a scope of a predetermined range.

12. The air-purifier system of claim 1, wherein the air purifier further comprises an aromatic substance that emits an aromatic odor when the aromatic substance is warmed.

13. The air-purifier system of claim 1 comprising a plurality of the air purifiers.
